(19) United States
(12) Patent Application Publication
(10) Pub. No.: US 2021/0315476 A1
(43) Pub. Date: Oct. 14, 2021

(54) Depth-independent method for in-vivo drug release monitoring and quantification based on magnetic particle imaging

(71) Applicant: The Board of Trustees of the Leland Stanford Junior University, Stanford, CA (US)

(72) Inventors: Bryan R. Smith, East Lansing, MI (US); Xingjun Zhu, Shanghai (CN); Jianfeng Li, Shanghai (CN)

(21) Appl. No.: 17/266,908

(22) PCT Filed: Aug. 22, 2019
(86) PCT No.: PCT/US2019/047656

§ 371 Date: Feb. 8, 2021

Related U.S. Application Data
(60) Provisional application No. 62/721,386, filed on Aug. 22, 2018.

(51) Int. Cl. A61B 5/0515; H01F 1/34; H01F 1/00; A61B 5/00; G01R 33/12; A61K 9/51; A61K 31/704

(52) U.S. Cl. CPC A61B 5/0515; H01F 1/344; H01F 1/0054; B82Y 5/00; G01R 33/1269; A61K 9/5153; A61K 31/704; A61B 5/4839

(57) ABSTRACT

A non-invasive drug release monitoring and quantification method and system is provided using magnetic particle imaging to monitor in vivo drug release. A living body is imaged with a magnetic particle imager after the living body has been injected with a nanocomposite composed of a biodegradable polymer shell layer containing a cluster of magnetic nanoparticles and a drug. A magnetic particle signal is detected and obtained which represents the release of magnetic nanoparticles from the PLGA shell layer, which is the result of a disassembly of the biodegradable polymer shell layer due to biological degradation of the biodegradable polymer shell layer in an acidic environment of the living body resulting in drug release and magnetic nanoparticle release. The release of the drug in the living body is quantified using a previously obtained reference linear relationship defined between the magnetic particle signal and the drug release rate.

### FIELD OF THE INVENTION

This invention relates to drug release monitoring and quantification methods and systems. In particular, the invention relates to in vivo drug release monitoring and quantification methods and systems using magnetic particle imaging (MPI).

### BACKGROUND OF THE INVENTION

Non-invasive in vivo drug release monitoring has gained increasing attention as it not only reduces side effects to patients but also increases efficacy by directly reflecting drug dosing in the living body. By quantitatively monitoring drug release process, it will help physician track drug doses timely and accurately in patients, which realizes quick adjustment of following doses to keep them in therapeutic window, thereby minimizing side effects and maximizing efficacy. Imaging approaches would be ideal for monitoring drug release process because they are intuitive diagnostic methods that are easily recognized and can give spatial information of drug distribution. Attempts have been made to monitor drug release by imaging modalities including optical imaging, photoacoustic imaging, or magnetic resonance imaging (MRI). However, the penetration depth of optical imaging and photoacoustic imaging in biological tissues is limited, which makes them unsuitable for deep tissue observation, precluding many clinical applications. MRI allows sufficient penetration depths for human use, but it's not linearly quantitative and unsuitable for quantitative measure of drug release. The present invention provides new methodology and systems to overcome the current shortcomings towards quantitative non-invasive drug monitoring.

### SUMMARY OF THE INVENTION

In-vivo drug release monitoring provides accurate and reliable information to guide drug dosing and potential side effects. Current image-based strategies for in-vivo monitoring have several merits such as non-invasiveness and visualization, but those imaging modalities in use (e.g. fluorescence imaging (FI) and magnetic resonance imaging (MRI)) remain inadequate because of the low tissue penetration depth (for FI) and difficulties in accurately quantifying the release rate (MRI).

It is critically important to develop a new method based on imaging to achieve in-vivo drug release monitoring with high penetration depth and the ability to quantify signals. Such a tool will allow physicians to spatially map the site(s) of active drug throughout the body, with knowledge of release rates. Magnetic particle imaging (MPI), employing superparamagnetic nanoparticles as the contrast agent, has large tissue penetration and linearly quantitative signal intensity, which is ideal for application to in-vivo drug release monitoring and the preferred imaging method in this invention. The linear quantitative signal intensity allows for extrapolation of existing measurements or prediction of future signal intensity based on existing (earlier time) measurements for in-vivo drug release monitoring.

The present invention is a nanocomposite which is composed of a clustered superparamagnetic Fe3O4 nanoparticles (SPIONs) core and a poly(lactide-co-glycolide acid) (PLGA) shell layer. By loading a chemo-drug, e.g. doxorubicin (DOX), into the PLGA shell, this nanocomposite can simultaneously serve as both a drug delivery system and a quantitative MPI tracer. As the PLGA shell degrades in the biological environment, the release process of DOX and the disassembly of clustered SPIONs core occur at the same time.

The disassembly of a clustered core results in an increase in the SPIONs' mobility, which can be quantitatively detected by enhancement of the MPI signal. By tracking the continuous changes in MPI signal intensity of the nanocomposites over time (including in real time) or time intervals, the drug release process is consequently monitored.

In this invention, we established calibration curves between drug release rate and MPI signal changes, ensuring that detection of drug release is linearly quantitative. Furthermore, we demonstrated the monitoring of in-vivo drug release by injecting nanocomposites into a human breast cancer model in mice and quantitatively tracking the drug release and induced cell kill.

The DOX-loaded superparamagnetic nanocomposite we designed serves as a drug delivery system as well as an MPI tracer. The as-prepared PLGA nanocomposite exhibits a sustained release of doxorubicin under a mild acidic environment (pH=6.5), such as that found within cells. We observed that the MPI signal changes in the nanocomposite display a linear correlation with the release rate of Doxorubicin in vitro. By utilizing this phenomenon, the relationship between MPI signal and drug release rate is established and quantitative monitoring of the release process in vitro is achieved. We then showed that in-vivo drug release monitoring for cancer therapy can be realized in a murine breast cancer model by injecting nanocomposite into mice. This invention allows for a new, better solution for in-vivo drug release monitoring compared to other available monitoring strategies, making it highly attractive for clinical use.

In terms of applications and use, embodiments of the invention can be in drug delivery to cancer (solid tumors), atherosclerosis, arthritis, neurodegeneration, and a variety of other localized diseases. This strategy has the benefits that it allows quantitative imaging of kinetics of drug release at the disease site, which will allow physicians to monitor whether cells in a particular diseased region are exposed to sufficient drug concentrations (e.g., if they found insufficient concentrations, more could be injected). Moreover, our approach can also show which other organs and regions of the body are receiving the drug and how much. This will ensure that physicians know which sorts of side effects to expect due to the drug and once known, may allow them to take immediate steps to reduce the off-target effects. Notably, while we have shown the ability to quantify the release of only one small molecule drug, a large variety of small molecule drugs are expected to be able to be loaded (any that can be loaded into our particles, and we have already loaded several others), and we anticipate that we will be able to monitor even larger therapeutic molecules such as peptides, nucleic acids, and potentially protein release analogously via in-vivo imaging.

Embodiments of this invention for in-vivo drug release monitoring, based on magnetic particle imaging, has essentially no limitation in penetration depth in the living body while retaining linearly quantifiable signals for determination of drug release. Moreover, MPI contrast is derived only from the injected nanoparticle contrast (yielding near-infinite contrast), therefore no other intrinsic structures can convolute the signal, unlike Mill and fluorescence. These advantages make the strategy provided herein superior to existing methods, including fluorescence imaging and magnetic resonance imaging.

In one embodiment, the invention is described as a method of using magnetic particle imaging to non-invasively monitor and quantify in vivo drug release. The method involves imaging a living body with a magnetic particle imager. The living body has been injected with a nanocomposite, which is composed of a biodegradable polymer shell layer containing a core of clustered magnetic nanoparticles and a drug that has been loaded into the nanocomposite.

In one example, the polymer shell layer is a poly(lactide-co-glycolide acid) (PLGA) shell layer. In one example, the drug is a hydrophobic drug. In another example, the drug is a chemotherapeutic drug, an anesthetic drug or an immune modulator drug, or more specifically the drug is doxorubicin. The magnetic nanoparticles are iron-oxide nanoparticles or more specifically, the iron-oxide nanoparticles are superparamagnetic Fe₃O₄ nanoparticles.

The method further involves detecting and obtaining from the magnetic particle imager a magnetic particle signal. The magnetic particle signal represents the release magnetic nanoparticles from the biodegradable polymer shell layer, which is the result of a disassembly of the biodegradable polymer shell layer due to biological degradation of the biodegradable polymer shell layer in an acidic environment of the living body resulting in a drug release and a magnetic nanoparticle release.

The method then further involves quantifying the drug release in the living body using a previously obtained reference linear relationship defined between the magnetic particle signal and the drug release rate. The method can be further extended to predict or extrapolate future drug release of the drug in the living body using the linear relationship.

In an exemplary embodiment, the linear relationship can be created by testing the PLGA nanoparticles (loaded with iron oxide nanoparticles) in pristine conditions in a test tube. The MPI signal can be measured at various time points and at the same times measured the doxorubicin that was released using spectroscopy. The MPI signal can be plotted as a function of released dox which turns out to be a linear relationship. A linear regression can be applied to obtain a line of best fit with an R-squared value.

Computer-implemented steps and methods executable by a computer processor are integrated within the method steps, where applicable and in conjunction with the magnetic particle imager and the data obtained from the magnetic particle imager.

### BRIEF DESCRIPTION OF THE DRAWINGS

FIGS. 1A-C show according to an exemplary embodiment of the invention the scheme of magnetic particle imaging (MPI) based drug release monitoring in vivo. Fe₃O₄@PLGA core-shell nanocomposites FIG. 1A loaded with doxorubicin (DOX) molecules FIG. 1B are prepared to serve as both MPI tracer and drug delivery system. When Fe₃O₄@PLGA were exposed to acidic environment or taken up by cells, the PLGA shell layer was degraded gradually, resulting in the disassembly of the clustered Fe₃O₄ core (large circles) and DOX (small circles) release at the same time. The clustered Fe₃O₄ core has low MPI signal intensity and the disassembly of the core can enhance MPI signal due to the increase of Brownian relaxation. By monitoring the change of MPI signal, the release process of drug molecules can thus be determined quantitatively.

FIGS. 2A-D show according to an exemplary embodiment of the invention characterizations of Fe₃O₄@PLGA-DOX nanocomposites. FIG. 2A: Scanning electron microscopy (SEM) image of Fe₃O₄@PLGA core-shell nanocomposites. FIG. 2B: SEM image of Fe₃O₄@PLGA core-shell nanocomposites after DOX loading (Fe₃O₄@PLGA-DOX). FIG. 2C: UV-Vis absorption spectra of Fe₃O₄@PLGA and Fe₃O₄@PLGA-DOX. Inset, photo of Fe₃O₄@PLGA (left) and Fe₃O₄@PLGA-DOX (right). FIG. 2D: Zeta potential of Fe₃O₄@PLGA and Fe₃O₄@PLGA-DOX.

FIGS. 3A-E show according to an exemplary embodiment of the invention drug release monitoring feature of Fe₃O₄@PLGA-DOX in solution. FIG. 3A: Time dependent DOX release behavior of Fe₃O₄@PLGA-DOX in pH=6.5 phosphate buffer. FIG. 3B: MPI signal change of Fe₃O₄@PLGA-DOX during the DOX release process in FIG. 3A. FIG. 3C: Correlation curve of DOX release percentage and MPI signal intensity. The curve exhibits a linear behavior which can be fitted by a function: Y=311.1X+3 0.91×10⁴ (R²=0.990). Inset, MPI images of Fe₃O₄@PLGA-DOX in solutions at different incubation time points. FIG. 3D: SEM images of Fe₃O₄@PLGA-DOX incubated with pH=6.5 phosphate buffer at different time points (12 and 48 h). FIG. 3E: Depth comparison between MPI and FI. Fe₃O₄@PLGA-DOX loaded with Cy5.5 was placed in a 96-well plate and covered with different thicknesses of chicken breast tissue. Both MPI and FI were conducted at each thickness of tissue slice.

FIGS. 4A-D show according to an exemplary embodiment of the invention Fe₃O₄@PLGA-DOX for MPI guided drug release monitoring in cell. FIG. 4A: Relative DOX release percentage from Fe₃O₄@PLGA-DOX in MDA-MB-231 cells. FIG. 4B: MPI signal change of Fe₃O₄@PLGA-DOX labelled MDA-MB-231 cells during the DOX release process. FIG. 4C: Correlation curve of DOX release percentage and MPI signal intensity in cells. The curve exhibits a linear behavior which can be fitted by a function: Y=117.3X+4.98×10⁴ (R²=0.991). Inset, MPI images of Fe₃O₄@PLGA-DOX labelled cells at different incubation time points. FIG. 4D: Cell apoptosis assay of MDA-MB-231 cells incubated with Fe₃O₄@PLGA-DOX at different time points.

FIG. 5 shows according to an exemplary embodiment of the invention Fe₃O₄@PLGA-DOX for MPI guided drug release monitoring in tumor bearing mice. FIG. 5A: MPI and CT merged images of MDA-MB-231 tumor bearing nude mouse with intratumoral injection of Fe₃O₄@PLGA-DOX. MPI signals are presented by pseudo-color. MPI signal intensity gradually increased with the extension of time after injection of Fe₃O₄@PLGA-DOX. FIG. 5B: Quantification of MPI signal intensity from tumor site in MDA-MB-231 tumor bearing nude mouse at a series of time points after injection (N=3 mice). FIG. 5C: Tumor sections of MDA-MB-231 tumor bearing nude mice injected with saline (Blank), Fe₃O₄@PLGA, DOX only and Fe₃O₄@PLGA-DOX, respectively. A TUNEL assay was used to evaluate apoptosis in tumors with different treatment and representative images are shown from N=3 mice per condition. Green signal (Alexa Fluor 488, AF488) indicates the apoptotic region in the tumor and red signal is doxorubicin (DOX) fluorescence.

### DETAILED DESCRIPTION

An ideal imaging modality for monitoring drug release would provide large imaging depths and linearly quantifiable signals. Magnetic particle imaging (MPI), a new non-invasive imaging modality employing superparamagnetic nanoparticles (SPNs) as tracers, would meet these specifications. In MPI scanning, an oscillating magnetic field with specific frequency and amplitude is applied to make SPNs generate unique higher harmonic signals which are collected by dedicated receiving coils to reproduce the spatial location, concentration and local environment of nanoparticles. MPI has near-infinite contrast as signals arise only from externally-administered SPNs tracer. It also has a large imaging depth which excludes the defects of optical and photoacoustic imaging due to the usage of the magnetic field as excitation. Moreover, by detecting signals of SPNs at higher harmonic frequency, MPI provides the information on local environment changes experienced by SPNs and accurately quantifies their concentrations. Based on the above-mentioned merits, as stipulated by the inventors, MPI has a broad prospect not only in disease diagnosis, but also in other biomedical applications requiring quantitative analysis. Hence, MPI is thus selected to quantify release of drug from our nanoparticle clusters.

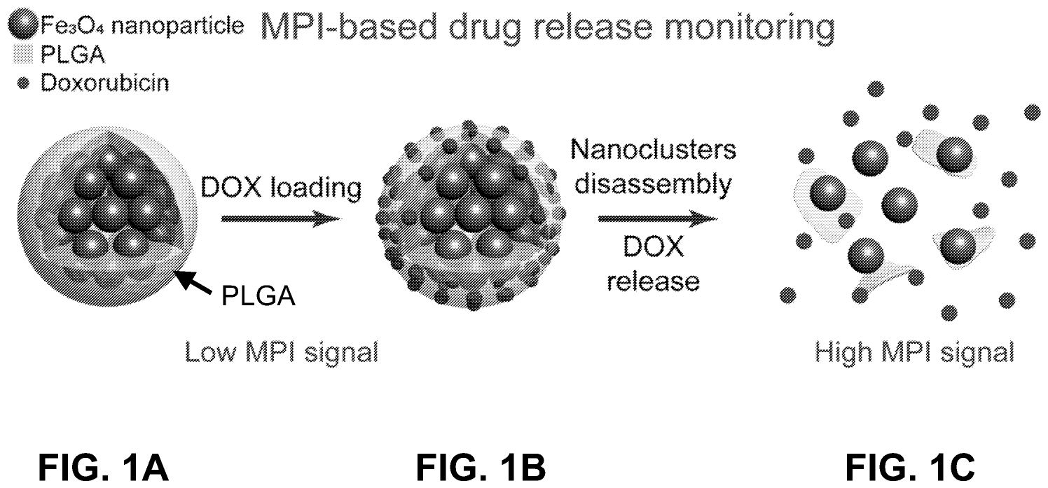

In this invention, we prepared a kind of nanocomposite which is composed of a clustered superparamagnetic Fe₃O₄ nanoparticles core and a poly(lactide-co-glycolide acid) (PLGA) shell layer (FIGS. 1A-C). By loading a commercially used chemodrug, doxorubicin (DOX), into the PLGA shell, this nanocomposite can serve as both a drug delivery system and a MPI tracer. As the PLGA shell degrades in biological environment, the release process of DOX and the disassembly of clustered SPIONs core occur at the same time. The disassembly of clustered core results in the increase of SPIONs' mobility, which can be reflected by the enhancement of MPI signal. By tracking the continuous changes of MPI signal intensity of the nanocomposites, drug release process is consequently being monitored. We established the calibration curve between drug release rate and MPI signal changes so that drug release can be measured quantitatively. Furthermore, we demonstrated the monitoring of in vivo drug release by injecting nanocomposites intratumorally in human breast cancer mice model. This invention is a method and system to achieve in vivo drug release monitoring that shows great potential in clinical translation.

Synthesis and Characterizations of Fe₃O₄@PLGA-DOX Nanocomposite

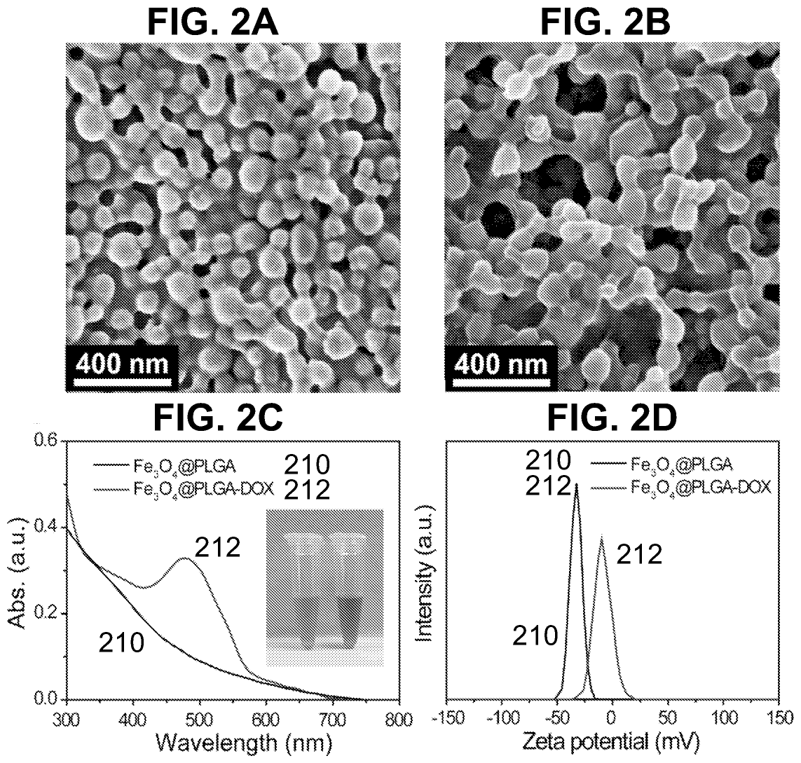

The nanocomposite with clustered Fe₃O₄ core and PLGA shell (Fe₃O₄@PLGA) was prepared by co-precipitation method. Scanning electron microscope (SEM) image of as-synthesized Fe₃O₄@PLGA showed that the nanocomposites have a nearly spherical morphology (FIG. 2A) with an average diameter of ˜105 nm. Doxorubicin (DOX) loaded nanocomposites (Fe₃O₄@PLGA-DOX) were prepared by mixing Fe₃O₄@PLGA (0.12 mg ml⁻¹) and DOX (0.143 μM) in PBS (pH=7.4) for 24 h. After DOX loading, the spheroid morphology of the nanocomposites remained (FIG. 2B). UV-Vis absorption spectroscopy also proved that DOX have been loaded in Fe₃O₄@PLGA due to the emergence of a new absorption band at 480 nm in the spectrum of Fe₃O₄@PLGA-DOX, indicating the existence of DOX molecules, when compared to its counterpart Fe₃O₄@PLGA (FIG. 2C). Zeta potential data of Fe₃O₄@PLGA and Fe₃O₄@PLGA-DOX confirm the success of the DOX loading process since the charge of the nanocomposites changed from −31.4 to −9.7 mV (FIG. 2D). This is because the protonated DOX molecules with positive charge had counteracted the negative charge in PLGA. Dynamic light scattering analysis revealed that Fe₃O₄@PLGA-DOX has an average hydrodynamic diameter of 122.7 nm (not shown). The loading ratio of DOX in Fe₃O₄@PLGA is determined by UV-Vis absorption spectroscopy. It was found that 82.9% of the original DOX (0.143 μM) was loaded in 0.12 mg ml⁻¹ of Fe₃O₄@PLGA.

Drug Release Monitoring with Magnetic Particle Imaging in Solution

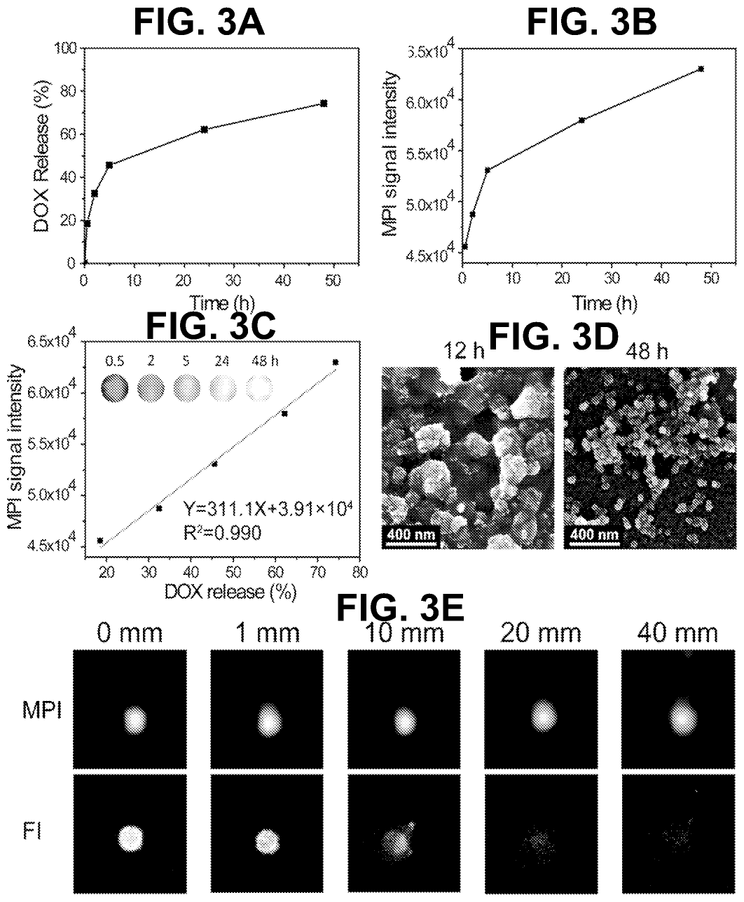

The successful synthesis of Fe₃O₄@PLGA-DOX prompted us to further investigate its application in drug release monitoring. As a proof of concept experiment, Fe₃O₄@PLGA-DOX nanocomposites were dispersed in pH=6.5 phosphate buffer solution to simulate the intracellular environment. Nanocomposite dispersion was kept at 37° C. for 48 h. The release of DOX at different time points were measured by UV-Vis absorption spectroscopy and the WI signals at the same time points were also recorded. As shown in FIG. 3A, the percentage of DOX released which determined by absorption spectra increased gradually as the extension of incubation time. The release rate exhibited a two-stage feature with initial fast release (from 0 to 5 h) and following slow release (from 5 h to 48 h). Interestingly, MPI signals of Fe₃O₄@PLGA-DOX changed correspondingly at different time points of the release process and the signal variation tendency came into line with DOX release behavior, which had an accelerated increase at the beginning followed by slow increase (FIG. 3B). When DOX release ratios and WI signals at each time points were integrated together, a linear-like correlation curve could be obtained. The function of this curve is expressed as Y=311.1X+3.91×10⁴ with an R²=0.99 (FIG. 3C). With this DOX release-MPI signal curve as calibration, monitoring of DOX release process can thus be successfully achieved by detecting the MPI signal changes of Fe₃O₄@PLGA-DOX nanocomposite. To understand the possible reason of the MPI signal change during drug release, we imaged the Fe₃O₄@PLGA-DOX particles with SEM (FIG. 3D). It can be clearly figured out that Fe₃O₄@PLGA-DOX particles underwent a degradation process during the incubation of pH=6.5 buffer solution. After 12 h of incubation, the morphology of Fe₃O₄@PLGA-DOX became more irregular and the surface of the particles were rough which may be due to the dissolution of PLGA shell layer. With even longer incubation (48 h), the size of particles decreased to ˜50 nm, indicating that the clustered structure of Fe₃O₄ nanoparticles was dissembled. Hence, it can be concluded that the increase of MPI signal intensities is due to the recovery of relaxation of Fe₃O₄ nanoparticles because of the degradation of PLGA. The movement of Fe₃O₄ nanoparticles coated with PLGA was restricted and thus the Brownian relaxation which is positively correlated to MPI signal intensity was suppressed. When PLGA was degraded, Fe₃O₄ nanoparticles was unfrozen, making relaxation enhance, and thus MPI signals increased.

To compare the imaging depth of MPI with fluorescence imaging, which is reported for drug release monitoring, we loaded Cy5.5 into Fe₃O₄@PLGA-DOX and imaged the nanocomposite by MPI and fluorescence imaging (FI). During the imaging, samples were covered with chicken breast in different thicknesses to investigate the imaging signal penetration in biological tissue. As shown in FIG. 3E, MPI gave an excellent tissue penetration of which the signals keep almost the same at all tissue depth conditions (from 0 to 40 mm). On the other hand, FI showed limited penetration capability of which the signals diminished substantially with the increase of tissue thickness. Hence, it can be safely concluded that MPI used for drug release monitoring will have indispensable advantages in deep tissue detection.

In Vitro Drug Release Monitoring with Magnetic Particle Imaging

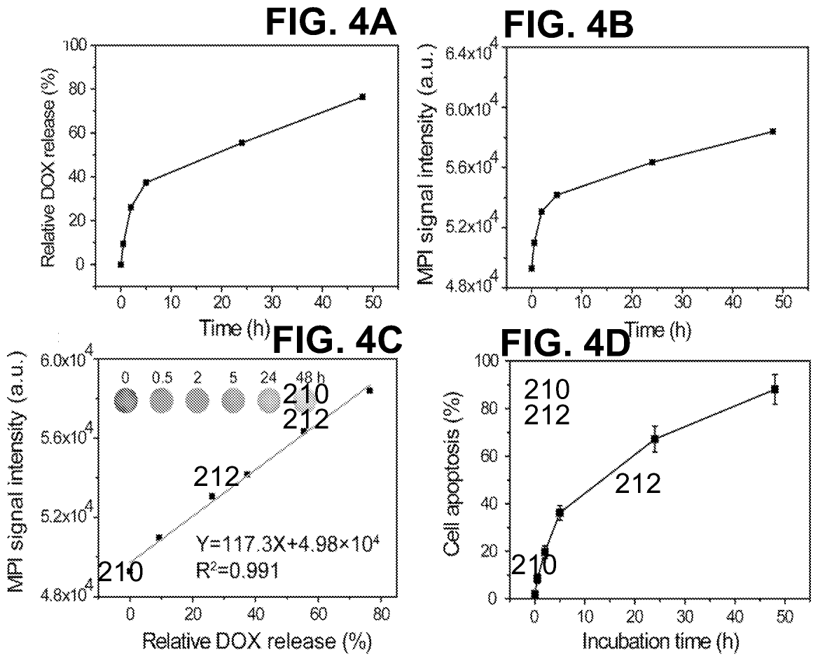

Based on the results of drug release monitoring by means of MPI in bulk solution, we further labelled Fe₃O₄@PLGA-DOX into cells and tried to investigate the feasibility of intracellular DOX release monitoring by MPI. Here, human breast adenocarcinoma cells, MDA-MB-231, were used to demonstrate the drug release monitoring experiment. After 3 h incubation of Fe₃O₄@PLGA-DOX contained media, MDA-MB-231 cells were tested by MPI at a series of time points to observe the change of signal intensity caused by the release process. The fluorescence of DOX molecules embedded in the PLGA shell layer was originally suppressed due to aggregation induced quenching effect, but the release process would gradually reduce the aggregation and thus recover the emission of DOX. Hence, fluorescence spectrometer was used to measure the emission of DOX during the release process to determine the drug release calibration curve with the combination of MPI signal changes. The emission intensity of DOX in cells increased continuously during the observation period of release process (not shown). We set the DOX emission detected from the first timepoint (0 h) in release process and the pure DOX solution with the same concentration of DOX in Fe₃O₄@PLGA-DOX contain media for cell incubation as 0% and 100% release, respectively. Afterwards, the relative DOX release at different time points was plotted based on the emission spectra, which is shown in FIG. 4A. The similar two-phase release behavior observed in the solution also occurred in the release process in cell level. Meanwhile, the MPI signal of cells labelled with Fe₃O₄@PLGA-DOX were also recorded. It is interesting that the variation trend of MPI signal is quite identical to that of DOX emission FIG. 4B. If the DOX emission and MPI signal were integrated together. A linear-like correlation curve could be also obtained. The function of this curve can be expressed as Y=117.3X+4.98×10⁴ with an R²=0.991 (FIG. 4C). With these results, we confirmed that drug release process can be successfully monitored in vitro by using Fe₃O₄@PLGA-DOX nanocomposite.

Furthermore, the therapeutic effect of Fe₃O₄@PLGA-DOX nanocomposite was evaluated by apoptosis assay in cell level. MDA-MB-231 cells were incubated with Fe₃O₄@PLGA-DOX for different time and were harvested for apoptosis test based on Annexin V expression on the outer leaflet membrane of cell. As shown in FIG. 4D, with the extension of incubating time, apoptosis effect was strengthened. The variation of cell apoptosis percentages with different incubation time of the nanocomposites meets the release behavior of DOX, showing fast increase at initial time points and slower rate in following time points.

In Vivo Drug Release Monitoring with Magnetic Particle Imaging

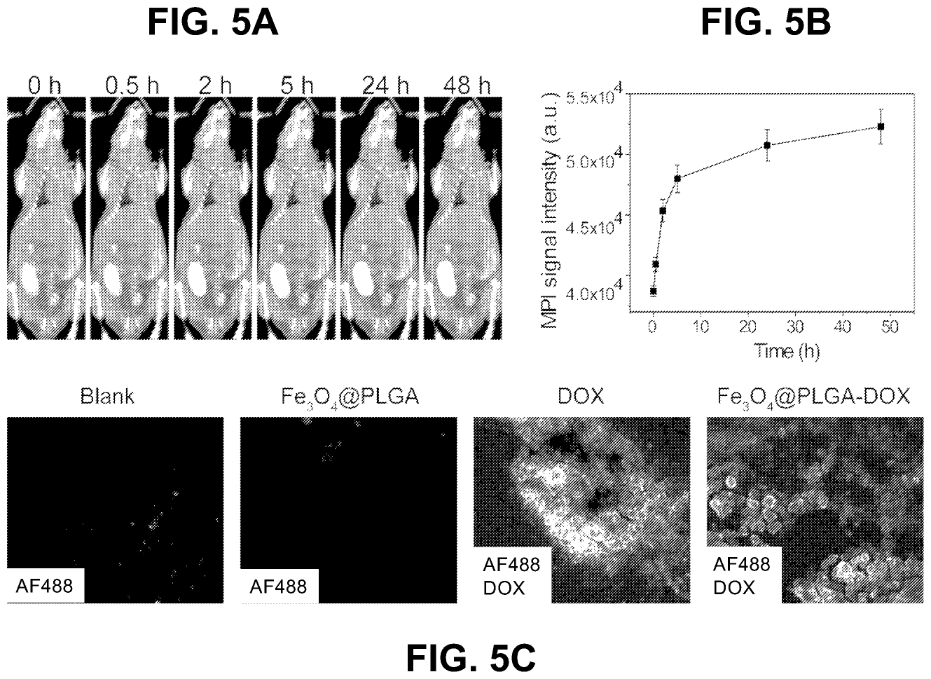

The successful monitoring of drug release process in vitro prompt us to investigate the possibility of monitoring drug release by MPI in living body. Here, we inject Fe₃O₄@PLGA-DOX intratumorally into orthotopic breast cancer (MDA-MB-231) mice model. MPI was conducted after injection of nanocomposite at different time points to see if there was any change in the signal intensity. As shown in FIG. 5A, MPI signals can be clearly observed in tumor region and the signal intensities increased gradually with the extension of observation time from 0 to 48 h. By integrating the signal intensities in tumor site, the signal changes were illustrated in FIG. 5B, from which we can see that MPI signal increased rapidly from 0 to 5 h and then experienced a slow rise after 5 h. The tendency of signal change was quite like the results observed in the in vitro study, which can be attributed to the release of DOX molecules from the nanocomposites. Based on the above results, we proved that drug release monitoring by MPI can be realized in vivo.

Furthermore, we evaluated the therapeutic efficacy of Fe₃O₄@PLGA-DOX to the tumor. Tumor tissues of the orthotopic breast cancer mice were harvested after injection of Fe₃O₄@PLGA-DOX for 48 h and then sectioned for TUNEL apoptosis assay. As demonstrated in the tumor section processed by TUNEL assay (FIG. 5C), tumor injected with Fe₃O₄@PLGA-DOX showed significant apoptosis which was detected by Alexa Fluor 488 (green signal). Meanwhile, the fluorescence signals can also be observed in the tissue section. Similarly, tumor injected with DOX solutions also exhibited apoptosis. On the other hand, tumor injected with Fe₃O₄@PLGA showed almost no apoptosis of which the effect is analogous to the injection of saline (Blank).

### CONCLUSION

In this invention, a type of nanocomposite, Fe₃O₄@PLGA-DOX, is shown to serve as both magnetic particle imaging (MPI) tracer and drug release monitoring system. The nanocomposite has a clustered superparamagnetic Fe₃O₄ nanoparticles core and a PLGA shell layer. Doxorubicin, as a model drug, is loaded into the PLGA layer. In acidic and intracellular environment, PLGA layer will be degraded resulting in the release of drug and the disassembly of cluster iron oxide core. The dissociation of the iron oxide core lead to the increase of MPI signal, which is correlated to the drug release percentage. By detecting the MPI signal changes, we realized real-time drug release monitoring both in vitro and in vivo. This is the first time that MPI is demonstrated to be used in biomedical detection, especially in living body. Due to the high tissue penetration and quantitative feature of MPI, drug release monitoring can be conducted without consideration of the tissue depth. Meanwhile, the quantity of drug released can be visualized by imaging results which is more direct and contains more information on spatial distribution.

Synthesis of Fe₃O₄@PLGA-DOX

Fe₃O₄ nanoparticles (1 mg) were mixed with 5 mg PLGA in 5 ml chloroform to form a mixture. The mixture was added into 40 ml aqueous solution containing 0.3% PVA and sonicated to form an emulsion. The emulsion was stirred at 40° C. overnight to evaporate chloroform and form nanoclusters. The obtained nanoclusters were washed with deionized water for 3 times and then dispersed in PBS solution. Doxorubicin (DOX) loaded on nanocluster were prepared by mixing nanoclusters (6.6 mg ml⁻¹) and DOX (143 μM) in PBS (pH=7.4) for 24 h. The resulted Fe₃O₄@PLGA-DOX were washed with PBS for 3 times.

Magnetic Particle Imaging of Fe₃O₄@PLGA-DOX for Drug Release Monitoring in Aqueous Solution

The as prepared Fe₃O₄@PLGA-DOX serves jointly as a drug delivery system and MPI-based drug-release monitoring agent. Nanoclusters were dispersed in 50 μl phosphate buffer (pH=6.5) to form a solution containing 30 μg ml⁻¹ Fe₃O₄ nanoparticles. The solutions in tubes were imaged for 6 timepoints (0, 0.5, 2, 5, 24, 48 h) with MPI. Meanwhile, UV-Vis absorption was used to measure the absorption spectra of DOX released in the solution.

Magnetic Particle Imaging of Fe₃O₄@PLGA-DOX for Drug Release Monitoring In Vitro

MDA-MB-231 cells were used to study the intracellular drug release. Cells were incubated with Fe₃O₄@PLGA-DOX at a concentration of 90 μg/ml for 6 timepoints (0, 0.5, 2, 5, 24, 48 h). MPI of cell samples was conducted at each timepoints. Meanwhile, fluorescent emission of cells samples was measured to determine the quantity of DOX released from the nanocomposites.

### Evaluation of Cell Apoptosis

Cell apoptosis was quantitatively evaluated by Annexin-V expression on the outer leaflet cell membrane with flow cytometry. Briefly, MDA-MB-231 cells were planted into a 6-well cell culture plate and were cultured at 37° C. and 5% CO₂ for 24 h. After that, cells were incubated with Fe₃O₄@PLGA-DOX at a concentration of 90 μg/ml for 6 timepoints (0, 0.5, 2, 5, 24, 48 h). and further cultured at 37° C. and 5% CO₂ for another 24 h. After that, cells were stained with Alexa Fluor 488 conjugated Annexin-V and then harvested for flow cytometry analysis.

### Tumour Xenografts

MDA-MB-231 cells were harvested by incubation with 0.05% trypsin-EDTA and then collected by centrifugation and resuspended in sterile phosphate buffer saline. Cells (5×10⁶ cells per mouse) were subcutaneously implanted into four-week-old female nude mice. Tumor bearing mice were ready to for bioimaging and programmed combination therapy was performed when the tumors reached an average diameter of 0.6 cm.

Magnetic Particle Imaging of Fe₃O₄@PLGA-DOX for Drug Release Monitoring In Vivo

MDA-MB-231 tumor bearing nude mice were injected intratumorally with Fe₃O₄@PLGA-DOX at a concentration of 90 μg/ml for 50 μl. The mice were imaged with MPI for 6 timepoints (0, 0.5, 2, 5, 24, 48 h). Meanwhile, mice were imaged by X-ray computed tomography at each timepoints to achieve the anatomy information.

### TUNEL Assay for Tumor Apoptosis Analysis

MDA-MB-231 tumors on nude mice were harvested and encapsulated in Tissue-Tek® O.C.T. and then frozen. Tumor tissues embedded in were sectioned by cryostat with a thickness of 2 μm for each slice. tissue slices were treated with Click-iT™ Plus TUNEL Assay (Alexa Fluor™ 488 dye) kit for apoptosis detection. Fluorescence images of tumor tissues were captured by fluorescent microscopy. Alexa Fluor™ 488 dye was excited by 488 nm laser and the emission collecting window was 500-540 nm. The signal of DOX were excited by 488 nm laser and emission was collected at 580-630 nm.

What is claimed is:

1. A method of using magnetic particle imaging to non-invasively monitor and quantify in vivo drug release, comprising:
(a) imaging a living body with a magnetic particle imager, wherein the living body has been injected with a nanocomposite, wherein the nanocomposite is composed of a biodegradable polymer shell layer containing a core of magnetic nanoparticles and a drug that has been loaded into the nanocomposite;
(b) detecting and obtaining from the magnetic particle imager a magnetic particle signal, wherein the magnetic particle signal represents a release of magnetic nanoparticles from the biodegradable polymer shell layer, which is the result of a disassembly of the biodegradable polymer shell layer due to biological degradation of the biodegradable polymer shell layer in an acidic environment of the living body resulting in a drug release and a magnetic nanoparticle release; and
(c) quantifying the release of the drug in the living body using a previously obtained reference linear relationship defined between the magnetic particle signal and the drug release rate.

2. The method as set forth in claim 1, further comprising predicting or extrapolating future drug release of the drug in the living body using the linear relationship.

3. The method as set forth in claim 1, wherein the biodegradable polymer shell layer is a poly(lactide-co-glycolide acid) (PLGA) shell layer.

4. The method as set forth in claim 1, wherein the drug is a hydrophobic drug.

5. The method as set forth in claim 1, wherein the drug is a chemotherapeutic drug, an anesthetic drug or an immune modulator drug.

6. The method as set forth in claim 1, wherein the drug is doxorubicin.

7. The method as set forth in claim 1, wherein the magnetic nanoparticles are iron-oxide nanoparticles.

8. The method as set forth in claim 1, wherein the iron-oxide nanoparticles are superparamagnetic Fe₃O₄ nanoparticles.
